SEQUENCE LISTING (27 sequences)

SEQ ID NO: 1 - protein, 15 aa, Artificial Sequence
  PNC-2 -ras-p21 residue 96-110
YREQIKRVKDSDDVP

SEQ ID NO: 2 - protein, 12 aa, Artificial Sequence
  PNC-7- ras-p21 residue 35-47
TIEDSYRQVVID

SEQ ID NO: 3 - protein, 16 aa, Artificial Sequence
  MRP- membrane resident peptide (penetratin)
KKWKMRRNQFVKVQRG

SEQ ID NO: 4 - protein, 13 aa, Artificial Sequence
  X13 peptide (control)
MPFSTGKRIMLGE

SEQ ID NO: 5 - protein, 29 aa, Artificial Sequence
  PNC-29- Control X13 peptide bound to penetratin
MPFSTGKRIMLGEKKWKMRRNQFVKVQRG

SEQ ID NO: 6 - protein, 15 aa, Artificial Sequence
  CD45- Control peptide from the CD-45 antigen
NAVFRLLHEHKGKKA

SEQ ID NO: 7 - protein, 31 aa, Artificial Sequence
  CD45-MRP- Control peptide from the CD-45 antigen linked to penetratin
NAVFRLLHEHKGKKAKKWKMRRNQFVKVQRG

SEQ ID NO: 8 - protein, 26 aa, Artificial Sequence
  PNC-28- Control peptide
ETFSDLWKLLKKWKMRRNQFVKVQRG

SEQ ID NO: 9 - protein, 31 aa, Artificial Sequence
  PNC-2 MRP- ras-p21 residue 96-110 bound to penetratin
YREQIKRVKDSDDVPKKWKMRRNQFVKVQRG

SEQ ID NO: 10 - protein, 28 aa, Artificial Sequence
  PNC-7 MRP- ras-p21 residue 35-47 bound to penetratin
TIEDSYRQVVIDKKWKMRRNQFVKVQRG

SEQ ID NO: 11 - protein, 14 aa, Artificial Sequence
  HIV-1 TAT(47-60)
YGRKKRRQRRRPPQ

SEQ ID NO: 12 - protein, 13 aa, Artificial Sequence
  D-TAT-transactivator of transcription cell penetrating peptide
GRKKRRQRRRPPQ

SEQ ID NO: 13 - protein, 7 aa, Artificial Sequence
  SV40-NLS
PKKKRKV

SEQ ID NO: 14 - protein, 16 aa, Artificial Sequence
  Nucleoplasmin-NLS
KRPAAIKKAGQAKKKK

SEQ ID NO: 15 - protein, 17 aa, Artificial Sequence
  HIV REV (34-50)
TRQARRNRRRRWRERQR

SEQ ID NO: 16 - protein, 15 aa, Artificial Sequence
  FHV (35-49) coat
RRRRNRTRRNRRRVR

SEQ ID NO: 17 - protein, 19 aa, Artificial Sequence
  BMV GAG (7-25)
KMTRAQRRAAARRNRWTAR

SEQ ID NO: 18 - protein, 13 aa, Artificial Sequence
  HTLV-II REX (4-16)
TRRQRTRRARRNR

SEQ ID NO: 19 - protein, 19 aa, Artificial Sequence
  CCMV GAG (7-25)
KLTRAQRRAAARKNKRNTR

SEQ ID NO: 20 - protein, 17 aa, Artificial Sequence
  P22N (14-30)- leader sequence
NAKTRRHERRRKLAIER

SEQ ID NO: 21 - protein, 22 aa, Artificial Sequence
  Lambda N (1-22)
MDAQTRRRERRAEKQAQWKAAN

SEQ ID NO: 22 - protein, 18 aa, Artificial Sequence
  Phi N (12-29)- Leader Sequence
TAKTRYKARRAELIAERR

SEQ ID NO: 23 - protein, 16 aa, Artificial Sequence
  Yeast PRP6 (129-124)
TRRNKRNRIQEQLNRK

SEQ ID NO: 24 - protein, 12 aa, Artificial Sequence
  Human U2AF
SQMTRQARRLYV

SEQ ID NO: 25 - protein, 26 aa, Artificial Sequence
  Human C-FOS (139-164)
KRRIRRERNKMAAAKSRNRRRELTDT

SEQ ID NO: 26 - protein, 28 aa, Artificial Sequence
  Human C-JUN (252-279)
RIKAERKRMRNRIAASKSRKRKLERIAR

SEQ ID NO: 27 - protein, 18 aa, Artificial Sequence
  p-vec
LLIILRRRIRKQAKAHSK

(19) United States
(12) Patent Application Publication
(10) Pub. No.: US 2010/0144636 A1
(43) Pub. Date: Jun. 10, 2010

(54) PEPTIDES DERIVED FROM RAS-P21 AND USES THEREFOR

(75) Inventors: Matthew R. Pincus, Brooklyn, NY (US); Josef Michl, Little Neck, NY (US); Victor Adler, Croton, NY (US)

Correspondence Address: HOFFMANN & BARON, LLP, 6900 JERICHO TURNPIKE, SYOSSET, NY, 11791, US

(73) Assignee: THE RESEARCH FOUNDATION OF STATE UNIVERSITY OF NEW YORK, ALBANY, NY (US)

(21) Appl. No.: 12/633,048

(22) Filed: Dec. 8, 2009

Related U.S. Application Data
(60) Provisional application No. 61/120,515, filed on Dec. 8, 2008.

(51) Int. Cl. A61K 38/16; C12Q 1/02; A61P 35/00

(52) U.S. Cl. 514/12; 435/29

(57) ABSTRACT

A method of treating ras-transformed and ras-related cancer, including: administering to a plurality cells, including ras-transformed cancer cells and normal cells, a peptide material having a membrane resident peptide and a ras-p21 component, the membrane resident peptide attached to the carboxyl or amino terminal end of the ras-p21 component.

### CROSS REFERENCE TO RELATED APPLICATIONS

This application claims priority to U.S. Provisional Patent Application No. 61/120,515 filed on Dec. 8, 2008, the contents of which is incorporated herein by reference in its entirety.

### FUNDING STATEMENT

This invention was made with government support under Grant No. CA 4250018, awarded by The National Cancer Institute. The Government may have certain rights in the invention.

### FIELD OF THE TECHNOLOGY

The present invention relates to the use of novel peptides as cancer treatments. Specifically, the present invention relates to two synthetic peptides which induce reversion of the cancer phenotype or necrosis of human cancer cells but have little to no effect on the viability or growth of normal cells.

### BACKGROUND

Oncogenic ras-p21 protein, but not its wild-type counterpart protein, induces malignant transformation of mammalian cell lines such as NIH 3T3 cells and has been implicated as a major causative factor in a high proportion of human solid tissue tumors. In Xenopus laevis oocytes, microinjection of oncogenic ras-p21 containing Val in place of Gly 12 (Val 12-p21 protein), but not wild-type ras-p21, has been found to induce oocyte maturation. Insulin induces oocyte maturation and requires activation of normal cellular ras-p21.

Since various cancers involve expression of Val 12-p21 protein, as well as other oncogenic proteins, it would be useful to be able to inhibit the mitogenic signal transduction pathways induced by expression of such proteins. For example, pancreatic cancer, over 90 percent of which expresses oncogenic ras-p21, is a nearly always fatal disease with a median survival time of only 80-90 days for a patient diagnosed with the disease. Pancreatic cancer is one of the more lethal forms of cancer in numbers of patients killed in the U.S. Less than 4% of patients are alive 5 years from the time of diagnosis.

### SUMMARY OF THE INVENTION

An aspect of the present invention provides a method of treating ras-transformed and other ras-related cancers. The method includes administering to a plurality cells, including ras-transformed cancer cells (or cancer cells transformed by overexpression of wild-type ras-p21 or transformed by overexpression of components on the oncogenic ras signaling pathway) and normal cells, a peptide material having a ras-p21 peptide sequence attached to a membrane resident peptide (MRP), or leader sequence, that allows transport of the ras-p21 peptides across the cell membrane, attached to the carboxyl or amino terminal end of the ras-p21 component. Where the ras-transformed cancer cells include a homozygous ras-p21 that is an oncogenic ras-p21, the administration step desirably results in necrosis of the ras-transformed cancer cells, and does not affect the normal cells. Where the ras-transformed cancer cells include a heterozygous mixture of wild-type ras-p21 and oncogenic ras-p21, the administration step desirably results in phenotypic reversion of the cancer cells to a plurality of non-cancerous cells, and does not affect these normal cells.

Another aspect of the present invention provides a method of treating ras-transformed cancer in a subject in need thereof. The method includes the steps of providing a subject having a ras-transformed cancer, where such cancer is either a homozygous ras-transformed cancer exhibiting only oncogenic ras-p21, or heterozygous ras-p21 cancer exhibiting both oncogenic ras-p21 and wild-type ras-p21; and administering to the subject a peptide material having a ras-p21 component and a membrane resident peptide (MRP). The MRP may also be called a “leader” that enables entry of the ras peptides into cells. In instances where the cancer is the homozygous ras-transformed cancer, necrosis of the cancer cells is an intended result. In instances where the cancer is the heterozygous ras-transformed cancer, reversion of the cancer cell phenotype is an intended result. It should be noted that whether or not the ras-transformed cancer cells are homozygous or heterozygous, normal, non-cancerous cells are not affected by the administration of the PNC-2-MRP (Seq. ID No. 9) or the PNC-7-MRP (Seq. ID No. 10).

Still another aspect of the present invention provides a method of determining a ras-transformed cancer. The method includes the steps of: providing a sample of cancer cells; administering to the sample cells an amount of an oncogenic ras-p21 blocking component; and observing a result thereof. If the observing step includes observing a level of LDH, then the cancer cells may be determined to be homozygous oncogenic ras-p21 transformed cancer. If the observing step includes observing phenotypic reversion, then the cancer may be determined to be composed of a heterozygous mixture of wild-type ras-p21- and oncogenic ras-p21-containing cells.

The various embodiments of the present invention may be better understood through a study of the following drawings and detailed descriptions.

### BRIEF DESCRIPTION OF THE DRAWINGS

FIG. 1 illustrates the effects of PNC-2-MRP (labeled p21 96-110) and PNC-7-MRP (labeled p21 35-47) on the growth of HT-1080 cells. Control was CD-45-MRP.

FIG. 2 is a graphical depiction of colony counts for HT-1080 cells treated with PNC-2-MRP and PNC-7-MRP peptides grown in soft agar. The positive control was HT-1080 cells treated with CD-45-MRP.

FIG. 3 graphically depicts the effects of PNC-2-MRP on cell growth of MIA-PaCa-2 cells. After 10 days, the cell count dropped to 0. Control was the CD-45-MRP.

FIG. 4 A depicts caspase expression as fraction of positive control (TNF-alpha-treated cells) in MIA-PaCa-2 cells treated with PNC-2-MRP and PNC-7-MRP. Controls were untreated MIA-PaCa cells (negative control) and MIA-PaCa-2 cells incubated for 45 min with tumor necrosis factor (TNF)alpha (Sigma) (20 ng/ml), known to induce apoptosis.

FIG. 4B depicts the LDH activity in medium for MIA-PaCa-2 cells: untreated (condition 1); treated with PNC-2-MRP (condition 2); treated with PNC-7-MRP (condition 3); treated with positive control PNC-28-MRP peptide (21) (condition 4).

FIG. 5 illustrates blots for the expression of JNK and phospho-JNK in both MIA-PaCa-2 (A, B) and HT-1080 cells (C, D) treated with PNC-2-MRP and PNC-7-MRP peptides.

### DETAILED DESCRIPTION OF THE INVENTION

### Abbreviations Used in the Present Application

| | |
|---|---|
| MAPK | Mitogen-activated protein kinase (also called ERK) |
| JNK | Jun-N-terminal kinase |
| TOPK | Lymphokine-activated killer T-cell-originated |
| | protein kinase (TOPK) |
| DYRKIA | Dual-specificity-tyrosine-phosphorylation-regulated |
| | kinase-IA |
| MRP | Membrane Resident Peptide |
| PNC-2 | Ha-ras peptide, 96-110 |
| PNC-7 | Ha-ras peptide, 35-47 |
| PNC-29 | Negative control peptide: cytochrome P-450 (called X13) |
| | attached to MRP |
| CD-45-MRP | Negative control peptide from the CD-45 antigen |
| | linked to MRP |
| LDH | Lactate dehydrogenase |

This invention relates to the surprising discovery by the present inventors of the selective mechanism of action of certain peptides; that when administered to cancer cells and normal non-cancerous cells, treatment of cancer cells occurs, but the normal non-cancerous cells are unaffected. This surprising result leads to the invention of the novel methods to treat cancer, and compositions of the present invention for treating the same. More specifically, this invention involves methods of treatment and compositions of synthetic peptide, non-peptide, and combination molecules for treating cancer, where the compounds selectively treat malignant and transformed cells only, even when administered to a mixture of normal non-cancerous cells and cancer cells. Also included are methods of treating cancer.

The term “patient” or “subject”, as used herein, may refer to a patient or patient population diagnosed with, or at risk of developing one or more forms of cancer. Also, as used herein, a subject may refer to a living animal, including mammals, which may be given cancer through transplantation or xenotransplanting which may be subsequently treated with the methods and compounds of the present invention or which have developed cancer and need veterinary treatment. Such subjects may include mammals, for example, laboratory animals, such as mice, rats, and other rodents; monkeys, baboons, and other primates, etc. They may also include household pets or other animals in need of treatments for cancer.

In accordance with the present invention, and as described in more detail below, the Applicants have surprisingly discovered that peptides designed from molecular modeling studies of the ras-p21 protein induce phenotype reversion of a pancreatic carcinoma cell line, but have no effect on normal pancreatic acinar cell growth. As has been discovered by the Applicants, two peptides described herein, designated PNC-2 (ras-p21 residues 96-110)-MRP [SEQ. ID NO. 1] and PNC-7 (ras-p21 residues 35-47)-MRP [SEQ. ID NO. 2], effectively block oncogenic ras-induced oocyte maturation, providing effective treatment thereof. The two inventive peptides described herein are especially useful in that they have been found to not block insulin-activated wild type ras-induced maturation. The instant discovery leads to forms of more effective and safer treatment of cancer.

Since various cancers involve expression mainly of Val 12-21 protein and other oncogenic forms that include single amino acid substitutions at Gly 12, Gly 13 and Gln 61, inhibition of these proteins, as well as phenotypic reversion of cancerous cells expressing these proteins represents a valuable cancer therapy. One out of every three solid tumors involves expression of Val 12-p21. For example, between 50-75% of colon cancers, greater than 90% of pancreatic cancers, 33% of all non-small cell carcinomas of the lung, 20% of gastric and bladder cancers, and a number of mesotheliomas involve expression of oncogenic ras-p21 protein. As explained herein, treatment of such cancer cells with the inventive PNC-2-MRP and/or PNC-7-MRP is useful in slowing the growth of or altogether killing these cancer cells, providing an effective treatment.

In accordance with the present invention, peptides bound to an MRP, including PNC-2-MRP [SEQ. ID NO. 9], PNC-7-MRP [SEQ. ID NO. 10], are useful in treating cancerous cells. In addition, analogs and derivatives of the inventive peptides, pharmaceutical preparations and methods of treatment using the inventive peptides, and pharmaceutical preparations based on analogs and derivatives of the inventive peptides may be implemented and administered to an individual for the treatment of a variety of cancers. Preferably, the cancers which are treated with the inventive peptides (including pharmaceutical compositions including the inventive peptides) are ras-induced and/or ras-related cancers. Treatment of ras-induced tumors by the compositions of the present invention may be used to convert malignant masses into benign ones, thereby stopping the progression of metastatic disease. In addition, treatment of such tumors may include killing malignant cells.

In one embodiment, the present invention provides a method of treating oncogenic ras-induced and ras-related cancer. The method includes administering to a plurality of cells, including ras-transformed (including ras-induced and/or ras-related) cancer cells, a peptide material having a membrane resident peptide and a wild-type ras-p21 component. Notably, in the inventive method, the plurality of cells may include normal (i.e., non-cancerous) cells, since such normal cells are substantially unaffected by the methods described herein.

In this method, the MRP may be attached to the carboxyl terminal end of the ras-p21 component. Other attachment sites may be used if desired. The Applicants have discovered that, where the ras-transformed cancer cells include a homozygous ras-p21 that is an oncogenic ras-p21, the administration step results in necrosis of the ras-transformed cancer cells. In addition, where the ras-transformed cancer cells are heterozygous for wild-type ras-p21 and oncogenic ras-p21, the Applicants have discovered that the administration step may result in phenotypic reversion of the cancer cells to a plurality of non-cancerous cells. It should be noted that in either case, normal cells are substantially unaffected by the administration step, while cancerous cells are either destroyed or reversed.

The present invention also provides a method of treating ras-transformed cancer and, in some cases, cancer cells containing only wild-type ras-p21 (including, but not limited to U-251 human astrocytoma cells), in a subject in need thereof. The method includes the steps of providing a subject having a ras-transformed cancer and/or a cancer containing only wild-type ras-p21, and administering to the subject a peptide material, the peptide material including a membrane resident peptide and an oncogenic ras-p21 component on the carboxyl or amino terminal end of the ras-p21 component.

As with above, the cancer may be either a homozygous ras-transformed cancer exhibiting only oncogenic ras-21, or it may be heterozygous ras-p21 cancer exhibiting both oncogenic ras-21 and wild-type ras-p21. In embodiments where the cancer is the homozygous ras-transformed cancer, the administration step may result in necrosis of the cancer cells. In embodiments where the cancer is the heterozygous ras-transformed cancer, the administering step may result in phenotypical reversion of the cancer cells. It should be noted that whether the ras-transformed cancer cells are homogenerous or heterogenerous, normal, non-cancerous cells are not affected by the administration of the inventive peptide materials described herein.

The present invention also provides a method of treating ras-transformed cancer cells in a subject in need thereof. The method includes the steps of providing a subject having a ras-transformed cancer, where such cancer is either a homogenous ras-transformed cancer exhibiting only oncogenic ras-p21 or heterozygous ras-p21 cancer exhibiting both oncogenic ras-p21 and wild-type ras-p21; and administering to the subject a peptide material having a MRP and an oncogenic ras-p21 component, the MRP attached to the carboxyl terminal end of the oncogenic ras-p21 component. This method may also include treatment of cancer cells that contain over-expressed wild-type ras-p21 or components of the oncogenic ras pathway.

As with the other methods of treatment described above, in embodiments where the cancer homozygously consists of oncogenic ras-transformed cells, the administration step may result in necrosis of the cancer cells. Also, in embodiments where the cancer is the heterozygous ras-transformed cancer, the administering step may result in phenotypically reverting the cancer cells. It should be noted, as with the above methods, that whether or not the ras-transformed cancer cells are homozygous or heterozygous, normal, non-cancerous cells are not affected by the administration of the inventive peptide materials described herein.

The peptide material further may include a membrane resident peptide (MRP) having the sequence KKWKMRRNQFVKVQRG (SEQ ID NO: 3) (sometimes referred to as penetratin) and/or a wild-type ras-p21 component. The wild-type ras-p21 component may be PNC-2, having the sequence YREQIKRVKDSDDVP (SEQ ID NO: 1). In other embodiments, the wild-type ras-p21 component may be PNC-7, having the sequence TIEDSYRQVVID (SEQ ID NO: 2). The peptide material to be administered include the wild-type ras-p21 component and the MRP attached thereto, resulting in either PNC-2-MRP (SEQ ID NO. 9) or PNC-7-MRP (SEQ ID NO. 10). The peptide material may be administered to a ras-transformed cancer cell, thereby resulting in the peptide material blocking oncogenic ras-p21-induced phosphorylation of Jun-N-terminal kinase (JNK) and, in the case of PNC-7-MRP, blocking activation of raf by oncogenic ras-p21.

The ras-gene-encoded p21 protein becomes oncogenic when arbitrary single amino acids are substituted for the normally occurring ones, such as Gly 12 and Gln 61, in the wild type protein. From the results of molecular modeling studies of the average conformations of the wild-type and multiple oncogenic form of ras-p21, it has been found that oncogenic amino acid substitutions induce stereotypical changes in the three-dimensional structures of specific domains of this protein. Two of these regions involve (i) residues 35-47 of the switch 1 domain and (ii) residues 96-110. The former residues occur in a domain that has been implicated in the interaction of ras-p21 with a number of target proteins including GTPase activating protein (GAP), raf, the guanine nucleotide exchange-promoting protein, SOS and phosphoinositol-3-hydroxy kinase (PI3K). The latter residues have been implicated in the interaction of ras-p21 with SOS and jun kinase (JNK) proteins.

The Applicants have synthesized both of the inventive peptides, referred to as PNC-7 and PNC-2, and assayed them for their abilities to block oncogenic ras-p21 induction of maturation of Xenopus laevis oocytes. Injection of oncogenic, but not wild-type ras-p21, into these oocytes induce oocyte maturation, i.e., completion of the second meiotic division, as measured by germinal vesicle membrane breakdown (GVBD). Insulin also induces oocyte maturation in a manner that requires activation of endogenous wild-type ras-p21.

The Applicants have also found that both PNC-7 and PNC-2 peptides block oncogenic (Val 12-containing) ras-p21-induced oocyte maturation, but have considerably less inhibitory activity on insulin-induced oocyte maturation. Thus, oncogenic and activated wild-type ras-p21 proteins utilize overlapping but distinct signal transduction pathways. Oncogenic ras-p21 has been found to induce oocyte maturation using pathways that require a direct interaction with jun-N-terminal kinase (JNK) and its substrate, the transcriptional activation factor, jun and activation of the raf-MEK-MAPK pathway. In contrast, insulin-activated wild-type ras-p21 appears not to depend completely on these two pathways. Thus, for example, glutathione-S-transferase, pi isozyme (GST-pi) has been identified as a selective inhibitor of JNK-induced phosphorylation of jun. Co-injection of GST-pi with oncogenic ras-p21 into oocytes results in complete inhibition of oocyte maturation while this protein has no effect on insulin-induced maturation. In addition, the MAPK phosphatase, MKP-1T4, that de-activates this kinase, completely blocks oncogenic ras-p21-induced oocyte maturation but has considerably less effect on insulin-induced maturation.

Furthermore, the Applicants have discovered that, in oocytes induced to mature with oncogenic ras-p21, levels of phosphorylated JNK and MAPK rise strongly over the course of oocyte maturation. As will be explained in detail below, these phosphorylations are blocked by the inventive peptides. In contrast, phosphorylation of both kinases increases to much lower and constant levels that do not correlate with extent of maturation in oocytes that have been-induced to mature with insulin.

Possible sites at which each of the two ras peptides may act have been identified. It has been found that PNC-2, for example, blocks the oncogenic ras-p21-JNK interaction with a dose-response curve that coincides with that obtained for its inhibition of oocyte maturation. This finding suggested that PNC-2 may block oncogenic ras selectively by inhibiting its interaction with JNK.

Similarly, it has been found that PNC-7 blocks the interaction of oncogenic ras-p21 with raf. Since both oncogenic and wild-type ras require raf activation, it was surmised that each ras protein interacts with raf in differing ways resulting in differential activation of downstream kinases. Since both oncogenic ras-p21- and insulin-induced oocyte maturation are raf-dependent but only oncogenic ras-induced maturation requires raf activation of MEK and MAPK, possible alternate raf targets for the wild-type pathway are desired but have heretofore been undetermined.

The Applicants have recently discovered, using a complete Xenopus gene array, that insulin induces significantly higher levels of expression of two kinases in insulin-matured oocytes: lymphokine-activated killer T-cell-originated protein kinase (TOPK), a direct raf target, and dual-specificity tyrosine-phosphorylation-regulated kinase-1A (DYRK1A), than in oncogenic ras-p21-matured oocytes. Downregulation of these kinases with specific SiRNA's results in complete blockade of insulin-induced maturation but does not affect oncogenic ras-p21-induced maturation. Thus, although raf activation is essential to signal transduction by oncogenic and wild-type ras-p21, the pathways appear to branch downstream of raf.

All of these findings suggest that oncogenic and wild-type ras-p21, while sharing common targets, utilize different signal transduction pathways. An important implication of this conclusion is that, in ras-transformed cancer cells, oncogenic ras-p21 may be selectively blocked in such a way as to leave wild-type mitogenic signal transduction pathways intact.

This conclusion is supported by studies of the effects of these two peptides on a ras-transformed pancreatic cancer cell line, called TUC-3. These cells may be derived from a rat pancreatic acinar cell line, called BMRPA1, which has been transfected with the ras-oncogene encoding Val 12-p21 protein. Each of the two inventive peptides (PNC-2 and PNC-7) were linked to a MRP that allows them to cross the cell membrane. Each peptide-MRP was incubated with BMRA1 and TUC-3 cells. Through this study, the Applicants discovered that both peptides induce complete reversion of TUC-3 cells to the untransformed phenotype within 2 weeks of incubation but had no effect on the viability or the growth of untransformed BMRPA1 cells. These results suggest that both peptides may block oncogenic ras-p21 selectively, and in cancer cells that possess intact wild-type ras or other regulated mitogenic pathways, these peptides may allow for normal cell growth. This study thus showed that the inventive peptides may be significantly important in treating cancerous cells while maintaining normal cells.

Also, in these studies with TUC-3 cells, the Applicants have prepared plasmids that encoded the sequences for PNC-2 and PNC-7 peptides, respectively. Transfection of these plasmids into TUC-3 cells also resulted in reversion of all of the cells to the untransformed phenotype. These results suggested that the effects of PNC-2-MRP and PNC-7-MRP were due to the ras peptides themselves.

The synthetic peptides of the present invention may be synthesized by a number of known techniques. For example, the peptides may be prepared using the solid-phase technique initially described by Merrifield in J. Am. Chem. Soc. 85:2149-2154 (1963), the contents of which are incorporated herein by reference. Other peptide synthesis techniques may be found, for example, in M. Bodanszky et al., Peptide Synthesis, John Wiley and Sons, 2d Ed., (1976), incorporated herein by reference, and other references readily available to those skilled in the art. Peptides may also be synthesized by solution methods as described in The Proteins, Vol. 1, 3d Ed., Neurath, H. et al., Eds., pp. 105-237, Academic Press, New York, N.Y. (1976), the contents of which are incorporated by reference herein. Appropriate protective groups for use in different peptide syntheses are described in the texts listed above as well as in J. F. W. McOmie, Protective Groups in Organic Chemistry, Plenum Press, New York, N.Y. (1973), the contents of which are incorporated by reference herein.

The peptides of the present invention may also be prepared by chemical or enzymatic cleavage from larger portions of the ras-p21 protein, or from the full-length ras-p21 protein. Likewise, MRPs for use in the synthetic peptides of the present invention may be prepared by chemical synthesis or enzymatic cleavage from larger portions or the full-length proteins from which such MRPs are derived.

Additionally, the peptides of the present invention may be prepared by recombinant DNA techniques. For most amino acids used to build proteins, more than one coding nucleotide triplet (codon) can code for a particular amino acid residue. This property of the genetic code is known as redundancy. Therefore, a number of different nucleotide sequences may code for a particular subject peptide. The present invention also contemplates use of a deoxyribonucleic acid (DNA) molecule that defines a gene coding for, i.e., capable of expressing a subject peptide or a chimeric peptide from which a peptide of the present invention may be enzymatically or chemically cleaved.

The presence of the membrane resident peptide allows the peptide to become membrane-active and to form well-defined pores in the cell membrane, causing membranolysis, which allow for extrusion of the intracellular contents from the interior of the cancer cell, resulting in the compromise of the integrity of the cell. Pores in the cancer cell membrane are formed as an immediate result of administration of the compound. After the pores are formed, treatment of the cancerous cell (including cell necrosis, or cell death) desirably results within a short time frame, ranging from 15 minutes to 48 hours. Pore formation in the plasma membrane, in turn, causes membranolysis of the cancer cells, as the holes in the cell walls start allowing excess fluid and free compound to rush into the cells which, simultaneously, start leaking cytoplasm into the surrounding environment. The compound entering the cells through the plasma membrane pores binds to the cell and forms pores in the mitochondrial membrane causing lysis of the mitochondria and the abrupt termination of cellular energy production. Once membranolysis starts, the cell may eventually undergo necrosis, or cell death, as a result of the treatment with the methods and compositions of the present invention. Therefore, the compositions and methods of treatment may have devastating effects on cancer cells. Not only do the methods and compositions of the present invention tend to eradicate cancer cells, they may be administered to cancer cells and healthy cells alike, and only cancer cells will be affected.

Thus, these methods may be used to treat a sample of cells containing both non-cancerous, normal cells, cancer cells, and combinations thereof. Such samples would include cell lines, tissue samples, tumors, and/or a subject having cancer in need of treatment. As the methods of treatment do not cause cell death of normal cells, these methods of treatment are focused on the cancer cells, irrespective of the mode of administration to the cell sample. Thus, these methods of treatment may be used for tumors or cancers that are widespread, inoperable, or otherwise not effectively treated with conventional means or combination therapies.

When administering the inventive peptide material to a plurality of cells, it may be desirable to administer the peptide material with a biocompatible material. Such material may increase the effectiveness of the administration by increasing solubility or dispersion of the material in situ. Also, when the peptide material is administered to a subject, preferably a mammal and more preferably a human, the peptide material may desirably be administered in a pharmaceutically acceptable carrier. As used herein, a “pharmaceutically acceptable carrier” includes any and all solvents, dispersion media, fillers, coatings, antibacterial and antifungal agents, isotonic agents and the like. The use of such media and agents are well known in the art.

The synthetic peptides of the present invention may be administered to a patient, preferably a human patient, as a pharmaceutical composition containing a therapeutically effective amount of at least one synthetic peptide according to the present invention together with a pharmaceutical acceptable carrier. The term “therapeutically effective amount” or “pharmaceutically effective amount” means the dose needed to produce in an individual, phenotypic reversion of neoplastic or malignant cells (i.e., transformation of cancer cells to benign or non-cancerous cells) and/or necrosis of malignant cells. The therapeutically effective amount may vary, and is dependent upon the individual characteristics of the patent in need of treatment, including the severity of the cancer growth as well as physical bodily characteristics of the patient.

Preferably, compositions containing one or more of the synthetic peptides of the present invention may be administered through any route (including, for example, intravenously) for the purpose of treating neoplastic or malignant disease such as cancer. As used herein, cancer includes any disease or disorder associated with uncontrolled cellular proliferation, survival, growth, or motility. Examples of different cancers which may be effectively treated using one or more the peptides of the present invention include but are not limited to: breast cancer, prostate cancer, lung cancer, cervical cancer, colon cancer, melanoma, pancreatic cancer and all solid tissue tumors (epithelial cell tumors) and cancers of the blood including but not limited to lymphomas and leukemias. Preferably, the cancer to be treated in accordance with the present invention is an oncogenic ras-induced cancer such as colon cancer, pancreatic cancer, non-small cell carcinoma of the lung, gastric cancer, bladder cancer and mesotheliomas.

Administration of the peptides of the present invention may be oral, intravenous, intranasal, intraperitoneal, intramuscular, intradermal or subcutaneous, by suppository or by infusion or implantation. When administered in such manner, the synthetic peptides of the present invention may be combined with other ingredients, such as carriers and/or adjuvants. There are no limitations on the nature of the other ingredients, except that they are desirably pharmaceutically acceptable, efficacious for their intended administration, preferably do not degrade the activity of the active ingredients of the compositions, and desirably do not impede importation of a subject peptide into a cell. The peptide compositions may also be impregnated into transdermal patches, or contained in subcutaneous inserts, preferably in a liquid or semi-liquid form which patch or insert time-releases therapeutically effective amounts of one or more of the subject synthetic peptides.

The pharmaceutical forms suitable for injection may include sterile aqueous solutions or dispersions and sterile powders for the extemporaneous preparation of sterile injectable solutions or dispersions. The ultimate solution form in all cases is preferably a sterile material in a fluid form, but other forms are capable of being used for administration. Typical carriers include a solvent or dispersion medium containing, e.g., water buffered aqueous solutions, i.e., biocompatible buffers, ethanol, polyols such as glycerol, propylene glycol, polyethylene glycol, suitable mixtures thereof, surfactants or vegetable oils. Sterilization may be accomplished utilizing any art-recognized technique, including but not limited to filtration or addition of antibacterial or antifungal agents. Examples of such agents include paraben, chlorbutanol, phenol, sorbic acid or thimerosal. Isotonic agents such as sugars or sodium chloride may also be incorporated into the subject compositions.

Production of sterile injectable solutions containing the subject synthetic peptides may be accomplished by incorporating one or more of the subject synthetic peptides described hereinabove in the required amount in an appropriate solvent with one or more of the various ingredients enumerated above, as required, followed by sterilization. Most desirably, the peptides are sterilized via filter sterilization. In order to obtain a sterile powder, the above solutions may be vacuum-dried or freeze-dried as necessary.

Inert diluents and/or edible carriers and the like may be part of the pharmaceutical compositions when the peptides are administered orally. The pharmaceutical compositions may be in any oral delivery system, including, for example, hard or soft shell gelatin capsules, be compressed into tablets, or may be in an elixir, suspension, syrup or the like.

The peptides of the present invention may thus be compounded for convenient and effective administration in pharmaceutically effective amounts with a suitable pharmaceutically acceptable carrier in a therapeutically effective dosage. Examples of a pharmaceutically effective amount include peptide concentrations in the range from about at least about 25 ug/ml to at least about 300 ug/ml. Other pharmaceutically effective amounts include peptide concentrations from about 50 to about 250 ug/ml, or from about 100 to about 200 ug/ml. It is understood that administration of the inventive peptides may be repeated as necessary to effectuate treatment, including repeated dosages hourly, daily, weekly, or any other pattern of administration.

The therapeutically effective amount of synthetic peptide to be used in the methods of the invention applied to humans may be different from patient to patient, and may be dependent upon several factors, including the stage of neoplastic disease, tumor size and aggressiveness, the presence or extent of metastasis, etc. In addition, the physiological characteristics of the subject may also be factors, including the individual's lean body weight, gender, and overall health. It can be generally stated, however, that the synthetic peptides of the present invention be administered in an amount of at least 10 mg per dose, or may be at least 50 mg per dose, may be at least 100 mg per dose, may be at least 500 mg per dose, or may be in an amount up to 1000 mg per dose. Since the peptide compositions of the present invention will eventually be cleared from the bloodstream, re-administration of the pharmaceutical compositions is indicated and preferred. Administration of the peptide may be in the form of a course of treatment, therapy, or combination therapy with other agents and/or medicaments as required.

The synthetic peptides of the present invention may be administered in a manner compatible with the dosage formulation and in such an amount as will be therapeutically effective. As explained above, systemic dosages may depend on the age, lean body weight, and condition of the patient and the administration route. An exemplary suitable dose for the administration to adult humans ranges from about 0.1 to about 20 mg per kilogram of lean body weight. Preferably, the dose is from about 0.1 to about 10 mg per kilogram of lean body weight.

Administering may include contacting. The term “contacting” refers to directly or indirectly bringing the cell and the compound together in physical proximity. The contacting may be performed in vitro or in vivo. For example, the cell may be contacted with the compound by delivering the compound into the cell through known techniques, such as microinjection into the tumor directly, injecting the compound into the bloodstream of a mammal, and incubating the cell in a medium that includes the compound.

Any method known to those in the art for contacting a cell, organ or tissue with a compound may be employed. Suitable methods include in vitro, ex vivo, or in vivo methods. In vitro methods typically include cultured samples. For example, a cell can be placed in a reservoir (such as a tissue culture dish), and incubated with a compound under appropriate conditions suitable for inducing treatment of cancer cells. Suitable incubation conditions can be readily determined by those skilled in the art.

Ex vivo methods typically include cells, organs or tissues removed from a mammal, such as a human. The cells, organs or tissues can, for example, be incubated with the compound under appropriate conditions. The contacted cells, organs or tissues are normally returned to the donor, placed in a recipient, or stored for future use. Thus, the compound is generally in a pharmaceutically acceptable carrier.

In vivo methods are typically limited to the administration of a compound, such as those described above, to a mammal, preferably a human. The compounds useful in the methods of the present invention are administered to a mammal in an amount effective in necrosing cancer cells for treating cancer in a mammal. The effective amount is determined during pre-clinical trials and clinical trials by methods familiar to physicians and clinicians.

An effective amount of a compound useful in the methods of the present invention, preferably in a pharmaceutical composition, may be administered to a mammal in need thereof by any of a number of well-known methods for administering pharmaceutical compounds. The compound may be administered systemically or locally.

The compounds useful in the methods of the invention may also be administered to mammals by sustained release, as is known in the art. Sustained release administration is a method of drug delivery to achieve a certain level of the drug over a particular period of time. The level typically is measured by serum or plasma concentration.

Any formulation known in the art of pharmacy is suitable for administration of the compounds useful in the methods of the present invention. For oral administration, liquid or solid formulations may be used. Some examples of formulations include tablets, capsules, such as gelatin capsules, pills, troches, elixirs, suspensions, syrups, wafers, chewing gum and the like. The compounds can be mixed with a suitable pharmaceutical carrier (vehicle) or excipient as understood by practitioners in the art. Examples of carriers and excipients include starch, milk, sugar, certain types of clay, gelatin, lactic acid, stearic acid or salts thereof, including magnesium or calcium stearate, talc, vegetable fats or oils, gums and glycols.

Formulations of the compounds useful in the methods of the present inventions may utilize conventional diluents, carriers, or excipients etc., such as those known in the art to deliver the compounds. For example, the formulations may comprise one or more of the following: a stabilizer, a surfactant, preferably a nonionic surfactant, and optionally a salt and/or a buffering agent. The compound may be delivered in the form of an aqueous solution, or in a lyophilized form. Similarly, salts or buffering agents may be used with the compound.

It should also be noted that treatment of cells may trigger an immunologic response in the body of a subject, often resulting in inflammation and/or swelling. As such, the methods of the present invention may also include the step of administering an anti-inflammatory agent or medicament. Many commonly accepted anti-inflammatory agents may be used, as is known in the art.

As will be explained in more detail in the Examples below, the Applicants have surprisingly discovered that the inventive ras peptides (PNC-2-MDP and PNC-7-MDP) are selective for blocking oncogenic ras pathways. Experiments conducted with PNC-2 and PNC-7, linked to a membrane-translocating MRP, against TUC-3 cells that are stably ras-transformed BMRPA1 pancreatic cancer cells that therefore contain both wild-type and oncogenic forms of ras-p21, resulted in a finding that the cells reverted to the untransformed phenotype. Since this result was reproduced in the same cells transfected with plasmids encoding either peptide, it was concluded that the ras-p21 sequences themselves induce reversion.

As will also be explained in more detail in the Examples below, the Applicants have surprisingly discovered that the two inventive ras peptides (PNC-2-MDP and PNC-7-MDP) also block human ras-transformed cancer cell growth. The abilities of these peptides to block human cancer cell growth are specific since control peptides, which contain the same MRP on their respective carboxyl termini, appear to have no effect on the growth of these cancer cell lines. The Examples set forth below indicate that the MRP itself is not responsible for the observed growth-inhibitory effects of PNC-2-MRP and PNC-7-MRP. In addition, these peptides exert their growth-inhibitory effects only on cancer cells since neither peptide is cytotoxic to untransformed cells in culture, including the untransformed normal BMRPA1 epithelial cell line.

In another aspect of the present invention, there may be provided a method of determining a ras-transformed cancer, and specifically what type of ras-transformed cancer exists. In one embodiment, the method includes the first step of providing a sample of cancer cells. The cancer cells may be naturally occurring or they may be modified. Any means to provide and maintain the cancer cells may be used, and desirably the cancer cells are maintained in such a fashion that they remain alive for testing and analysis. Next, an amount of an oncogenic ras-p21 blocking component is administered to the sample cells. Any blocking component as described above may be used, and may include the two inventive peptides described above. The amount to be administered may be correlated to the amount of sample cells provided, in such an amount that a result would be obtained.

After the blocking component is administered, the user may observe the resulting effect of such administration. In instances where the observing step includes observing a level of LDH, then the sample cancer cells may be determined to be homozygous oncogenic ras-p21 transformed cancer. In instances where the observing step includes observing phenotypic reversion, then the cancer may be determined to be composed of a heterozygous mixture of wild-type ras-p21- and oncogenic ras-p21-containing cells. Once determination has been made as to the type of cancer cells, proper and effective treatment of those cells may commence. Treatment may include, for example, administration of one of the two inventive peptides described herein.

### Mechanisms for Peptide Induction of Cancer Cell Growth Arrest

As shown in more detail in the Examples below, both PNC-2-MRP and PNC-7-MRP peptides induced phenotypic reversion of ras-transformed HT-1080 human fibrosarcoma cells as revealed by the inability of treated cells to grow on soft agar. This result correlates well with previous results on k-ras-transformed TUC-3 rat pancreatic cancer cells. After 2 weeks of treatment of these cells with either peptide, the cells were found to adopt the wild-type phenotype; unlike untreated TUC-3 cells, these treated cells failed to grow in nude mice. On the other hand, surprisingly, both the PNC-2-MRP and PNC-7-MRP peptides induced cell death, and not the phenotypic reversion, of MIA-PaCa-2 human pancreatic cancer cells. This result indicates either that there may be a change in the mechanism of inhibition by these peptides, of cancer cell growth in this cell line or that this cell line may lack alternate “rescue” pathways allowing for normal cell growth.

### EXAMPLES

### Investigation of Two Peptides from the ras-p21 Protein

An investigation into the effects of two peptides (referred to herein as the “target peptides”) from the ras-p21 protein, corresponding to residues 35-47 (PNC-7) and 96-110 (PNC-2), on two ras-transformed human cancer cell lines was conducted. The two ras-transformed human cancer cell lines included HT1080 fibrosarcoma and MIAPaCa-2 pancreatic cancer cells. The effects of these peptides on U-251 astrocytoma cells that overexpress wild-type ras-p21 and JNK were also studied. In prior studies, the Applicants discovered that both peptides block oncogenic, but not insulin-activated wild-type, ras-p21-induced oocyte maturation. However, as explained in the Examples herein, it has now been surprisingly discovered that when linked to a transporter MRP, these peptides induce reversion of oncogenic ras-transformed rat pancreatic cancer cells (TUC-3) to the untransformed phenotype.

The two target peptides and a control peptide, all of which were linked to a MRP, were incubated with each cell line. Cell counts were obtained over several weeks. The cause of cell death was determined by measuring caspase in the cells as an indicator of apoptosis and, in the cell culture medium, lactate dehydrogenase (LDH) as marker of necrosis. Since both of the target peptides block the phosphorylation of jun-N-terminal kinase (INK) in oocytes, cell lysates of the two cancer cell lines for the levels of phosphorylated INK were blotted to determine if the peptides reduced these levels.

As will be explained in more detail below, it was discovered that both of the target peptides, but not the control peptide linked to the MRP, induce phenotypic reversion of the HT-1080 cell line, but cause tumor cell necrosis of the MIA-PaCa-2 cell line. On the other hand, neither of the target peptides has any effect on the viability of an untransformed, normal pancreatic acinar cell line, BMRPA1. It was discovered that, while total JNK levels remain constant during peptide treatment, phosphorylated JNK levels decrease dramatically, consistent with the mechanisms of action of the target peptides. As such, it was concluded that the target peptides block tumor but not normal cell growth. The blocking of tumor growth is achieved by blocking oncogenic ras-p21-induced phosphorylation of INK, an essential step on the oncogenic ras-p21-protein pathway. The target peptides may therefore be useful as anti-tumor agents.

Four peptides, two ras-p21 (PNC-2 [SEQ ID NO. 1] and PNC-7 [SEQ ID NO. 2]) and two control peptides (X13 [SEQ ID NO. 4] and CD45 [SEQ ID NO. 6]), were synthesized by solid phase methods. Each peptide contained, on its carboxyl terminal end, the transmembrane-transporting peptide sequence, KKWKMRRNQFVKVQRG [SEQ ID NO. 3]. All four peptides were shown to be >95% pure by mass spectroscopy. The sequences of these peptides are as follows:

1. ras-p21 96-110 (PNC-2)-MRP:

[SEQ ID NO. 9]

YREQIKRVKDSDDVP-KKWKMRRNQFVKVQRG;
2. ras-p2 1 35-47 (PNC-7)-MRP:

[SEQ ID NO. 10]

TIEDSYRQVVID-KKWKMRRNQFVKVQRG;
3. Control X13-MRP (PNC-29) peptide:

[SEQ ID NO. 5]

MPFSTGKRIMLGE-KKWKMRRNQFVKVQRG;
4. Control CD45-MRP:

[SEQ ID NO. 7]

NAVFRLLHEHKGKKA-KKWKMRRNQFVKVQRG.

The X13 sequence in control sequence (number 3) is from cytochrome P450. In addition, for the LDH assays for tumor cell necrosis (see below), another control peptide was employed: PNC-28, known to induce tumor cell necrosis, whose sequence is ETFSDLWKLL-KKWKMRRNQFVKVQRG [SEQ ID NO. 8].

The effect of each peptide-MRP was studied in the following cell lines: HT1080 (human fibrosarcoma) and MIAPaCa-2 (human pancreatic cancer) cell lines, obtained from the American tissue type and cell collection (ATCC) (Manassas, Va.); U-251 (human astrocytoma) cells, obtained from Dr. D. Weinstin (GliaMed, NY).

### Procedures

### Cell Cultures.

MIAPaCa-2 cells were grown in Dulbecco's modified Eagle's medium (GIBCO) supplemented with heat inactivated 10% fetal calf serum, 20 mM glutamine, penicillin (100 units/ml), streptomycin (100/ug/ml) and plated on a six-well plate with density no more then 20,000 cells/well in 3 ml culture medium. HT1080 cells were cultured in RPMI 1640 medium supplemented with 10% heat-inactivated fetal calf serum, 100 units/ml of penicillin and 100 ug/ml streptomycin and plated on six-well plates with a density of no more then 6,000 cells/well in 3 ml culture medium. BMRPA1 normal rat pancreatic acinar cells were grown as described previously.

Incubation of Peptides with Cells

All cells (approximately 2×10⁴) were incubated in their respective media (see above) for 24 h after which the media were replaced with the same media containing one of the peptides at a concentration of 100 μg/ml. Over the subsequent time course of each experiment, the medium was changed every 48 h, but always contained peptide at the same concentration. Cells were observed daily for 1-3 weeks for changes in morphology and growth characteristics. After selected time periods, cells were stained with trypan blue or assayed using the MTT assay as described previously to assess cell counts. These experiments were performed at least three times for each cell line.

### Growth of Cells in Soft Agar

To assess whether cells that appeared by morphology to have reverted to the untransformed phenotype after treatment with a peptide, we assessed whether the cells would grow on soft agar. In these experiments, performed three times, the cells were isolated, replated, allowed to grow to confluence in 10% FCS-DMEM and then trypsinized. At this time, 1×10⁴ cells were then mixed with 0.37% bactagar (Difco Laboratories, USA) and then added to culture plates containing solidified 0.6% bactagar medium. After the agar solidified, the plates were incubated at 37° C. for 2 weeks after which they were subjected to cell counts using the MTT assay. Colony counts were obtained directly from observing the agar.

### Caspase and LDH Assays

To detect if peptide-induced cell death occurred by apoptosis or necrosis, the Clontech caspase (CPP32) activity assay for elevated caspase was performed as described previously. In addition, to detect if significant cell necrosis occurred, the CytoTox96 assay for LDH released into the cell culture medium was used as also described previously. As a positive control for LDH release, the CytoTox96 assay was performed on the media of MIA-PaCa-2 cells that were incubated with 100 ug/ml PNC-28 peptide for 24 h.

### Immunoblots for Total and Phosphorylated JNK

Blots for total and phosphorylated JNK were performed as described previously for the following cells: HT1080 prior to incubation and after 2 weeks incubation with PNC-2-MRP and PNC-7-MRP; and MIA-PaCa-2 cells prior to incubation and after 2 weeks incubation with these two peptides. Approximately, 2×10⁶ cells were twice washed with cold PBS and lysed by adding the lysis buffer. Samples of lysates containing constant amounts of protein (either 50 or 75 μg) were subject to SDS PAGE on a 12% resolving gel and the proteins then electrophoretically transferred onto nitrocellulose membranes overnight at 4° C.; the membranes were then blocked with non-fat dry milk in Tris-buffered saline with 1% Tween-20 (TBS-T) (pH 7.6) and were then incubated with the appropriate anti-kinase antibodies as follows: (1) anti-JNK polyclonal antibody, which recognizes both JNK-1 and JNK-2, diluted 1:1,000; (2) anti-phospho JNK (JNK-P) phosphorylated at positions Thr 183 and Tyr 185 (diluted 1:800). All incubations were performed for 12 h at 4° C., after which the membranes were washed three times with TBS-T and incubated with anti-rabbit secondary antibody at 1:4,000 dilution. Detection was accomplished using the ECL chemiluminescence detection kit.

### Results

Effects of PNC-2-MRP and PNC-7-MRP on ras-transformed HT-1080 Fibrosarcoma Cells

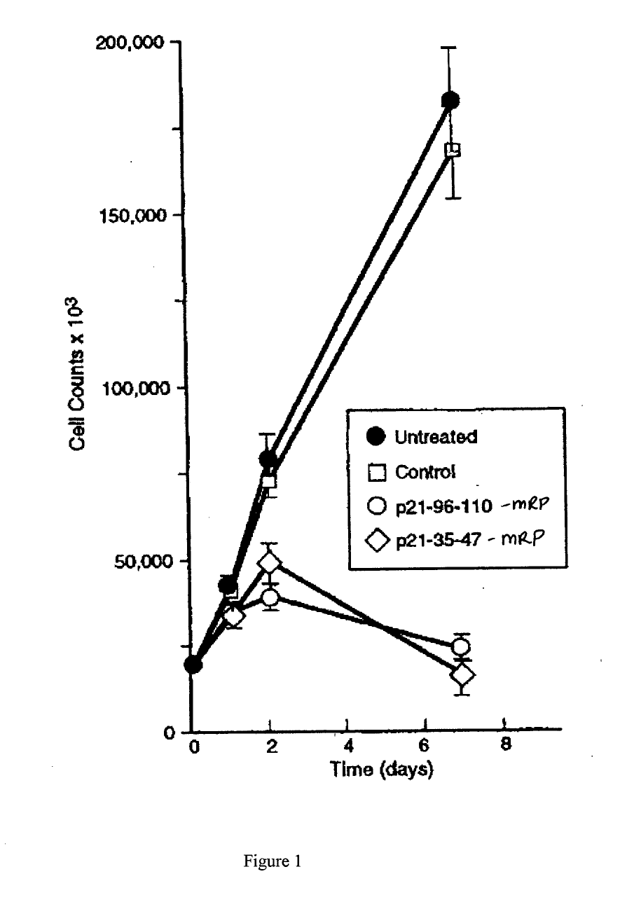

HT-1080 is a ras-transformed fibrosarcoma cell line. As can be seen in FIG. 1, when these cells were incubated with either PNC-2- or PNC-7-MRP peptides, after a small increase in tumor cell number up to 48 h, there was a dramatic decrease in cell count after 1 week. This count stabilized below the initial level and remained stable thereafter for a three-week observation period. As also shown in FIG. 1, control peptides such as X13-MRP (PNC-29) [SEQ ID NO. 5] and CD-45-MRP [SEQ ID NO. 7], had no effect on tumor cell growth which was the same as for untreated cells (FIG. 1). In addition, incubation of BMRPA1 cells with PNC-2-MRP [SEQ ID NO. 9], PNC-7-MRP [SEQ ID NO. 10] or PNC-29 control peptide [SEQ ID NO. 5] had no effect either on cell growth or viability. These results suggest that inhibition of tumor cell growth by both PNC-2-MRP and PNC-7-MRP peptides is peptide-specific and that the growth-inhibitory effects of these peptides is specific for cancer cells.

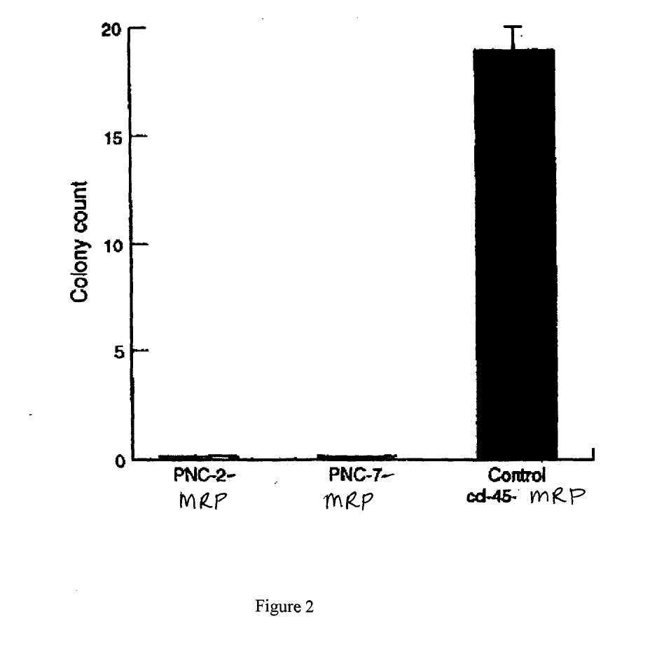

Since the cell count stabilized for HT-1080 cells that were treated with PNC-2-MRP or PNC-7-MRP (FIG. 1), it was explored whether the cells may have reverted to the untransformed phenotype as was found for TUC-3 cells treated with each of these peptides. These treated cells were plated in soft agar. As a control, the control peptide-treated cells were also plated in soft agar. FIG. 2 shows that the control peptide-treated cells formed multiple colonies, while the cells treated with either of the target ras peptides did not form any colonies. This result indicates that both target peptides induce loss of a critical characteristic of transformed cells. Coupled with the Applicant's finding that both target peptides block the growth of these cells, it is concluded that the target peptides induce phenotypic reversion of this cell line to non-transformed cells.

### Effect of ras Peptides on MIA-PaCa-2 Pancreatic Cancer Cells

Since the Applicants found that both of the target ras peptides induce complete phenotypic reversion of the TUC-3 rat pancreatic cancer cell line, and since oncogenic ras is known to be a major causative factor in over 90% of human pancreatic cancer, the study was applied to a ras-transformed human pancreatic cancer cell line, MIA-PaCa-2. The procedure was completed as set forth above, applied to MIA-PaCa-2.

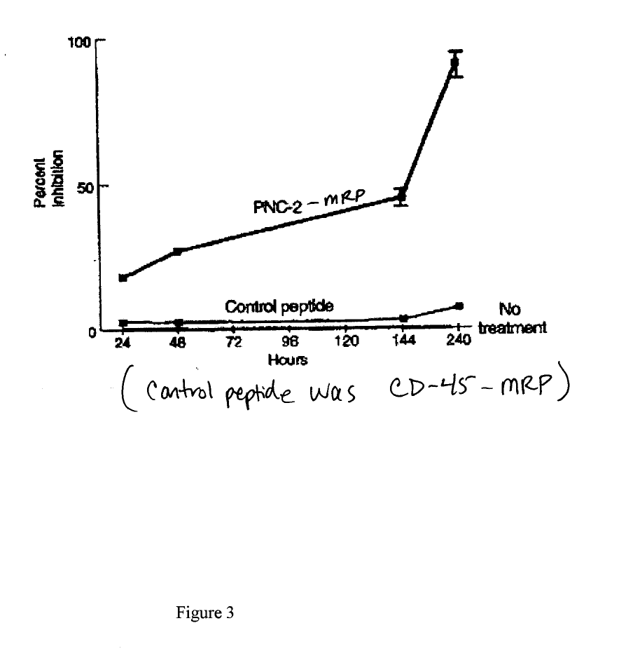

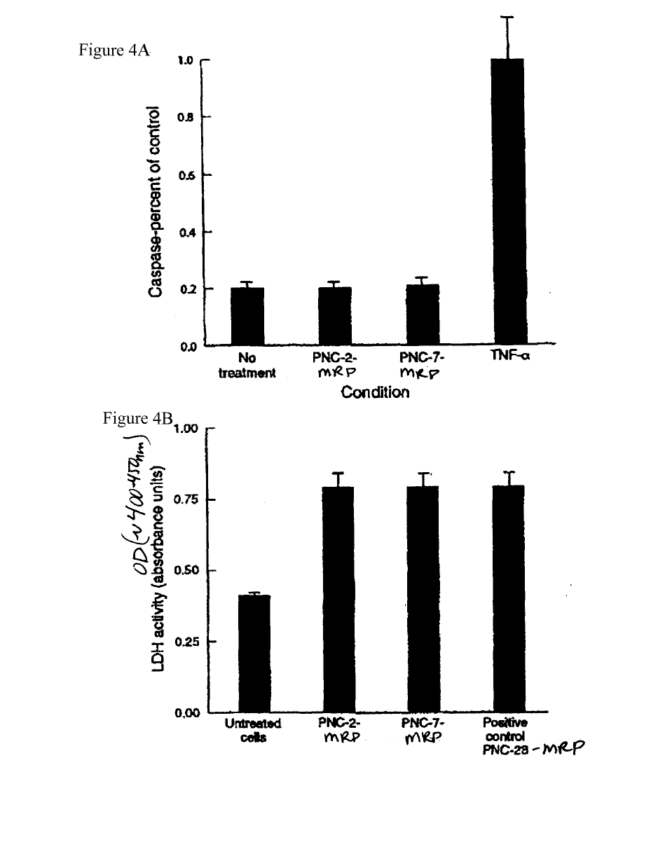

As shown in FIG. 3, both of the target peptides, but not the negative control peptide, induced total inhibition of tumor cell growth. When the cells were examined after 2 weeks of treatment with either of the target peptides, it was found that no viable cells remained i.e., there was total cell death. In order to explore the general cause of tumor cell death, the cells were assayed for caspase, a marker for apoptosis. In addition, the culture medium was assayed for lactate dehydrogenase (LDH) as a marker for tumor cell necrosis. As shown in FIG. 4A, caspase expression, while markedly elevated in cells treated with TNF-alpha that is known to induce apoptosis, is not elevated above control background levels in cells treated with either of the target ras-derived peptides. On the other hand, as shown in FIG. 4B, LDH is released from cells treated with either target ras peptide, but is twofold lower in the media of untreated cells or cells treated with PNC-29-MRP control peptide (not shown). These results suggest that both of the inventive PNC-2-MRP and PNC-7-MRP peptides induce tumor cell necrosis of ras-transformed MIA-PaCa-2 cells.

As shown in FIG. 4A neither peptide induces increased caspase expression in this cell line, eliminating this mechanism for induction of cell death. In other studies on peptides derived from p53, it has been found that these peptides induce tumor cell necrosis evidenced by release of the cytosolic enzyme, LDH, into the culture medium due presumably to cell membrane breakdown. As shown in FIG. 4B, both peptides induce the release of high levels of LDH into the culture medium. These results indicate that both peptides induce tumor cell necrosis.

### Effects of Peptide Treatment on JNK Expression and Phosphorylation

In prior studies on oocytes, it has been found that, in oocytes that were induced to mature by microinjection of oncogenic ras-p21, there was an early, strong induction of JNK and MAPK phosphorylation that increased as maturation progressed. In contrast, in oocytes induced to mature with insulin, that activates wild-type ras-p21, phosphorylation of JNK became observable at a later time and remained at a much lower level than that observed in oncogenic ras-p21-matured oocytes. When oncogenic ras-p21 was co-injected with either of the two inventive ras peptides, maturation was blocked, and phosphorylation of both JNK and MAPK was strongly diminished.

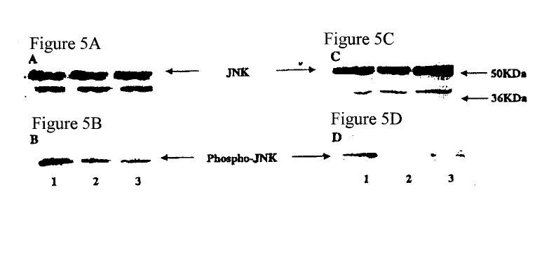

Since both of the target peptides have been found to induce tumor cell necrosis of ras-transformed MIA-PaCa-2 cells, it was examined whether these two target peptides cause diminished phosphorylation of JNK as was observed in the oocyte system. As shown in FIG. 5, expression of phosphorylated JNK is high in the untreated cells.

With reference to FIG. 5, in A, all lanes were blotted for total JNK. The Lanes are as follows: Lane 1, untreated MIA-PaCa-2 cells; Lane 2, MIA-PaCa-2 cells treated for 48 h with PNC-2-MRP; Lane 3, MIA-PaCa-2 cells treated for 48 h with PNC-7-MRP.

In B (FIG. 5), all lanes were blotted for phospho-JNK as described above. The Lanes are as follows: Lane 1, untreated MIA-PaCa-2 cells; Lane 2, MIA-PaCa-2 cells treated for 48 h with PNC-2-MRP; Lane 3, MIA-PaCa-2 cells treated for 48 h with PNC-7-MRP.

In C (FIG. 5), all lanes were blotted for total JNK: The Lanes are as follows: Lane 1, untreated HT-1080 cells; Lane 2, HT-1080 cells treated for 48 h with PNC-2-MRP; Lane 3, HT-1080 cells treated for 48 h with PNC-7-MRP.

In D (FIG. 5), all lanes were blotted for phospho-JNK as described above. The Lanes are as follows: Lane 1, untreated HT-1080 cells; Lane 2, HT-1080 cell treated for 48 h with PNC-2 MRP; Lane 3, HT-1080 cells treated for 48 h with PNC-7-MRP.

In cells treated with either PNC-2-MDP or PNC-7-MRP, while the expression of total JNK remained the same as in untreated cells, the level of phosphorylated JNK was markedly diminished. These results indicate that both inventive peptides block activation of JNK by oncogenic ras-p21 that may be a causative factor in tumor cell death.

As shown in FIG. 5, HT1080 and MIA-PaCa cells have high levels of phosphorylated JNK as was originally found in oocytes that were microinjected with oncogenic ras-p21. As was also found in oocytes, both peptides cause large decreases in phosphorylated JNK in both HT1080 and MIA-PaCa-2 ras-transformed human cancer cell lines. Since oncogenic ras appears to require JNK as a critical downstream target and since both PNC-2 and PNC-7 block its activation, it is believed that this blockade is critical to the inhibitory effects of both peptides.

It was found that the two cell lines respond differently to the target peptides, i.e., phenotypic reversion or necrosis, respectively. One possible difference may lie in the presence of alternate or “rescue” pathways. If both peptides block signal transduction by oncogenic ras, continued cell growth would then depend on the availability of alternate pathways such as the TOPK/DYRK1A pathway in oocytes. HT1080 cells are known to be heterozygous for oncogenic ras. If wild-type ras in these cells can function via alternate pathways such as may be available in the oocyte system or ones similar to them, normal cell growth may result. On the other hand, if such pathways are lacking, such as may exist in homozygously ras-transformed cells, all cell growth may cease, leading to cell death. Since MIA-PaCa-2 cells are homozygous for oncogenic ras-p21, this latter phenomenon may take place as a result of peptide treatment.

Effects of PNC-2-MRP and PNC-7-MRP on Cancer Cells that Do Not Contain Mutant ras-p21.

Both peptides were incubated with a human astrocytoma cell line referred to as U-251. It was found that both peptides kill all of these cells (1×10⁶) within 1 week at a concentration of 125 ug/ml. These cells are known to contain wild-type ras-p21 that is overexpressed. It has also been found that these cells express high levels of JNK. Functional interactions of Raf and MEK with Jun-N-terminal kinase (JNK) result in a positive feedback loop on the oncogenic Ras signaling pathway. Thus it was concluded that the inventive peptides, PNC-2-MRP and PNC-7-MRP, also are capable of killing cancer cells that express high levels of wild-type ras-p21 and/or high levels of JNK.

TABLE I—depicts the sequences and SEQ ID NOS: of materials referenced herein and employable with the methods of the present invention.

TABLE I

| | | SEQ |
|---|---|---|
| | | ID |
| Name | Sequence | NO: |
| PNC-2 | YREQIKRVKDSDDVP | 1 |
| PNC-7 | TIEDSYRQVVID | 2 |
| MRP | KKWKMRRNQFVKVQRG | 3 |
| X13 peptide | MPFSTGKRIMLGE | 4 |
| (control) | | |
| PNC-29 | MPFSTGKRIMLGEKKWKMRRNQFVKVQRG | 5 |
| CD45 | NAVFRLLHEHKGKKA | 6 |
| CD45-MRP | NAVFRLLHEHKGKKAKKWKMRRNQFVKVQRG | 7 |
| PNC-28 | ETFSDLWKLLKKWKMRRNQFVKVQRG | 8 |
| PNC-2 MRP | YREQIKRVKDSDDVPKKWKMRRNQFVKVQRG | 9 |
| PNC-7 MRP | TIEDSYRQVVIDKKWKMRRNQFVKVQRG | 10 |

Although the above table lists one preferred MRP used herein, other MRP's which may be useful in the present invention may take the form of the following sequences, listed below in Table 2.

TABLE 2

| | | SEQ |
|---|---|---|
| | | ID |
| Name | Sequence | NO: |
| HIV-1 TAT (47-60) | YGRKKRRQRRRPPQ | 11 |
| D-TAT | GRKKRRQRRRPPQ | 12 |
| SV40-NLS | PKKKRKV | 13 |
| Nucleoplasmin-NLS | KRPAAIKKAGQAKKKK | 14 |
| HIV REV (34-50) | TRQARRNRRRRWRERQR | 15 |
| FHV (35-49) coat | RRRRNRTRRNRRRVR | 16 |
| BMV GAG (7-25) | KMTRAQRRAAARRNRWTAR | 17 |
| HTLV-II REX (4-16) | TRRQRTRRARRNR | 18 |
| CCMV GAG (7-25) | KLTRAQRRAAARKNKRNTR | 19 |
| P22N (14-30) | NAKTRRHERRRKLAIER | 20 |
| Lambda N (1-22) | MDAQTRRRERRAEKQAQWKAAN | 21 |
| Phi N (12-29) | TAKTRYKARRAELIAERR | 22 |
| Yeast PRP6 | TRRNKRNRIQEQLNRK | 23 |
| (129-124) | | |
| Human U2AF | SQMTRQARRLYV | 24 |
| Human C-FOS | KRRIRRERNKMAAAKSRNRRRELTDT | 25 |
| (139-164) | | |
| Human C-JUN | RIKAERKRMRNRIAASKSRKRKLERIAR | 26 |
| (252-279) | | |
| p-vec | LLIILRRRIRKQAKAHSK | 27 |

Other membrane resident peptides may also be used. Such sequences are described, e.g., in Scheller et al. (2000) Eur. J. Biochem. 267:6043-6049, and Elmquist et al., (2001) Exp. Cell Res. 269: 237-244, the contents of which are incorporated by reference herein. In addition, U.S. patent application Ser. No. 11/825,242 filed on Jul. 5, 2007 (claiming priority to Jun. 1, 2004 and May 31, 2005), published as Publication No. US 2008/0153754, is incorporated herein by reference in its entirety. Additionally, the publication entitled, “Two peptides derived from ras-p21 induce either phenotypic reversion or tumor cell necrosis of ras-transformed human cancer cells”, authored by M. R. Pincus et al., Cancer Chemother. Pharmacol., v. 62, no. 3, August 2008, (E-Pub Dec. 8, 2007) is incorporated herein by reference in its entirety.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

What is claimed is:

1. A method of treating ras-transformed cancer, comprising:
(i) providing a plurality of cells, said plurality of cells comprising:
(A) ras-transformed cancer cells and normal cells;
(B) cancer cells that either overexpress wild-type ras-p21 and/or jun-N-terminal kinase (JNK) and/or its target, jun and normal cells;
(C) cancer cells lines that express oncogenic forms or that overexpress wild-type forms of any component like raf, MEK or MAPK that is essential to the oncogenic ras-p21 pathway;
(D) normal cells; and
(E) any combination of A, B, C, and D; and
(ii) administering a peptide material having a membrane resident peptide and a ras-p21 component, said membrane resident peptide attached either to the carboxyl or amino terminal end of said ras-p21 component.

2. The method of claim 1, wherein the oncogenic ras-transformed cancer cells comprise a homozygous ras-p21 that is an oncogenic ras-p21.

3. The method of claim 2, wherein the administration step results in necrosis of the oncogenic ras-transformed cancer cells.

4. The method of claim 1, wherein the ras-transformed cancer cells comprise a heterozygous mixture of wild-type ras-p21 and oncogenic ras-p21.

5. The method of claim 4, wherein the administration step results in phenotypic reversion of the cancer cells to a plurality of non-cancerous cells.

6. The method of claim 1, wherein the peptide material comprises a membrane resident peptide having the sequence KKWKMRRNQFVKVQRG (SEQ ID NO:3).

7. The method of claim 1, wherein the peptide material comprises a ras-p21 component PNC-2 having the sequence YREQIKRVKDSDDVP (SEQ ID NO: 1).

8. The method of claim 1, wherein the peptide material comprises a ras-p21 component PNC-7 having the sequence TIEDSYRQVVID (SEQ ID NO: 2).

9. The method of claim 1, wherein the peptide material blocks oncogenic ras-p21-induced phosphorylation of Jun-N-terminal kinase (JNK).

10. A method of treating ras-transformed cancer in a subject in need thereof, comprising the steps of:
(i) providing a subject having a ras-transformed cancer, where such cancer is either a homozygous ras-transformed cancer exhibiting only oncogenic ras-p21 or heterozygous ras-p21 cancer exhibiting both oncogenic ras-p21 and wild-type ras-p21;
(ii) administering to the subject a peptide material having a membrane resident peptide and a ras-p21 component, said membrane resident peptide attached to the carboxyl terminal or amino end of said ras-p21 component; and
(iii) treating said ras-transformed cancer; wherein if said cancer is said homozygous oncogenic ras-transformed cancer, necrosing said cancer cells, and if said cancer is said heterozygous oncogenic ras-transformed cancer, phenotypically reverting said cancer cells.

11. The method of claim 10, wherein the peptide material further comprises a membrane resident peptide having the sequence KKWKMRRNQFVKVQRG (SEQ ID NO:3).

12. The method of claim 10 wherein the membrane resident peptide comprises a positively charged sequences that transport peptides and proteins across cell membranes selected from the group consisting of: (Arg)₈, TAT of HIV1, D-TAT, R-TAT, SV40-NLS, nucleoplasmin-NLS, HIV REV, FHV coat, BMV GAG, HTLV-II (REX), CCMV GAG, P22N, Lambda N, Delta N, yeast PRP6, human U2AF, human C-FOS, human C-JUN, yeast GCN4, p-vec, and combinations thereof.

13. The method of claim 10, wherein the peptide material further comprises a ras-p21 component PNC-2 having the sequence YREQIKRVKDSDDVP (SEQ ID NO: 1).

14. The method of claim 10, wherein the peptide material further comprises a ras-p21 component PNC-7 having the sequence TIEDSYRQVVID (SEQ ID NO: 2).

15. The method of claim 10, wherein the peptide material blocks oncogenic ras-p21 induced phosphorylation of Jun-N-terminal kinase (JNK).

16. The method of claim 10, wherein the peptide material is in a pharmaceutically acceptable carrier.

17. A method of determining a ras-transformed cancer, comprising:
(i) providing a sample of cells, said cells comprising cancer cells;
(ii) administering to said cells an amount of an oncogenic ras-p21 blocking component; and
(iii) observing a result thereof.

18. The method of claim 16, further comprising the step of determining that said cancer cells comprise a homozygous oncogenic ras-p21 transformed cancer, if said observing step comprises observing a level of LDH.

19. The method of claim 16, further comprising the step of determining that said cancer cells comprise a heterozygous oncogenic ras-p21 transformed and wild-type ras-p21 cancer, if said observing step comprises observing phenotypic reversion.

20. The method of claim 16, wherein the administering step comprises administering PNC-2 with MRP attached to a carboxyl or amino terminal end of said PNC-2.

21. The method of claim 16, wherein the administering step comprises administering PNC-7 with MRP attached to a carboxyl or amino terminal end of said PNC-7.

22. The method of claim 20, wherein the MRP comprises the sequence KKWKMRRNQFVKVQRG (SEQ ID NO:3).

23. The method of claim 20, wherein the MRP comprises a positively charged sequences that transport peptides and proteins across cell membranes selected from the group consisting of: (Arg)₈, TAT of HIV1, D-TAT, R-TAT, SV40-NLS, nucleoplasmin-NLS, HIV REV, FHV coat, BMV GAG, HTLV-II (REX), CCMV GAG, P22N, Lambda N, Delta N, yeast PRP6, human U2AF, human C-FOS, human C-JUN, yeast GCN4, p-vec, and combinations thereof.

24. The method of claim 21, wherein the MRP comprises the sequence KKWKMRRNQFVKVQRG (SEQ ID NO:3).

25. The method of claim 21, wherein the MRP comprises a positively charged sequences that transport peptides and proteins across cell membranes selected from the group consisting of: (Arg)₈, TAT of HIV1, D-TAT, R-TAT, SV40-NLS, nucleoplasmin-NLS, HIV REV, FHV coat, BMV GAG, HTLV-II (REX), CCMV GAG, P22N, Lambda N, Delta N, yeast PRP6, human U2AF, human C-FOS, human C-JUN, yeast GCN4, p-vec, and combinations thereof.
